(19) United States
(12) Patent Application Publication
(10) Pub. No.: US 2016/0120896 A1
(43) Pub. Date: May 5, 2016

(54) POLYSACCHARIDE COMPOSITIONS AND RELATED METHODS

(71) Applicant: MOMENTA PHARMACEUTICALS, INC., Cambridge, MA (US)

(72) Inventors: Jing Wang, Malden, MA (US); Elaine Y. Sun, Boston, MA (US); Kristaq Vezuli, Worcester, MA (US); Daniela Beccati, Watertown, MA (US); Birgit Schultes, Arlington, MA (US); Jennifer Ozug, Norwood, MA (US); Miroslaw Lech, North Chelmsford, MA (US); Fei Yu, Quincy, MA (US)

(21) Appl. No.: 14/893,515

(22) PCT Filed: May 27, 2014
(86) PCT No.: PCT/US14/39538

§ 371 Date: Nov. 23, 2015

Related U.S. Application Data
(60) Provisional application No. 61/828,106, filed on May 28, 2013.

(51) Int. Cl. A61K 31/727; G01N 24/08

(52) U.S. Cl. CPC A61K 31/727; G01N 24/08

(57) ABSTRACT

The disclosure provides LMWH preparations and methods of analyzing LMWH preparations having at least one chain having a glycol split uronic acid residue (UG) in the preparation for structural signatures, and methods of producing such preparations based upon the analysis.

### BACKGROUND OF THE INVENTION

The invention relates to compositions and methods related to glycol split containing low molecular weight heparin (LMWH).

### SUMMARY OF THE INVENTION

In one aspect, described herein are pharmaceutical compositions comprising a LMWH preparation having at least one chain having a glycol split uronic acid residue (U_{G}), wherein formic acid is detectable in the composition but at less than 2%, 1%, 0.5%, 0.3%, 0.2%, 0.1%, 0.05% as determined by 1D-NMR and/or C₂H₆O₃ is detectable in the composition but at less than 2%, 1%, 0.5%, 0.3%, 0.2%, 0.1%, 0.05% as determined by 2D-NMR.

In one aspect, described herein are pharmaceutical compositions comprising a LMWH preparation having at least one chain having a glycol split uronic acid residue (U_{G}), wherein formic acid is detectable in the composition at a level between 0.0001-2% (e.g., 0.001-2%, 0.01-2%, 0.1-2%, 1-2%, 0.05-2%, 0.5-2%, 0.5-1%), e.g., NMR (e.g., 1D-NMR, 2D-NMR) and/or C₂H₆O₃ is detectable in the composition at a level between 0.0001-2% (e.g., 0.001-2%, 0.01-2%, 0.1-2%, 1-2%, 0.05-2%, 0.5-2%, 0.5-1%), e.g., NMR (e.g., 1D-NMR, 2D-NMR).

In some embodiments, the LMWH preparation comprises: (a) a weight average chain molecular weight between 3,500 and 8,000 Da, (e.g., 5,000-8,000 Da); (b) anti-Xa activity of less than 50 IU/mg (e.g., less than 40, 30, 20, 15, 10, 5, 2, 1, 0.5, 0.1, 0.01, 0.001 IU/mg) and anti-Ha activity of less than 50 IU/mg (e.g., less than 40, 30, 20, 15, 10, 5, 2, 1, 0.5, 0.1, 0.01, 0.001 IU/mg); (c) greater than 5% (e.g., 10, 20) and less than 50% (e.g., less than 40, 30, 25, 20, 10) glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation contains less than, e.g., 1%, 0.5%, 0.1% of formic acid when stored at about 4° C. (e.g., 2-6° C., 3-4° C., 3-6° C., 4-5° C., 4-6° C.) for 3 months or more, (e.g., 4, 5, 6, 7, 8, 9, 10, 11, 12, 24, 36 months or more). In some embodiments, the LMWH preparation is an M402 preparation. In some embodiments, the LMWH preparation the M402 preparation is Necuparinol.

In some embodiments, the LMWH preparation the LMWH preparation comprises a polysaccharide of Formula (I)

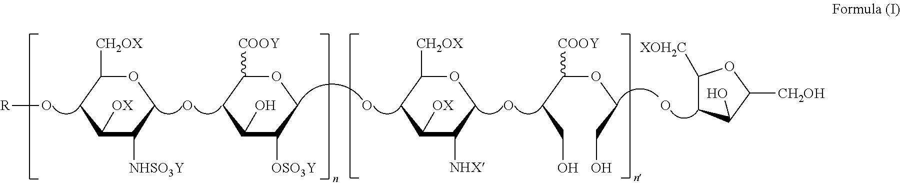

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and
R is

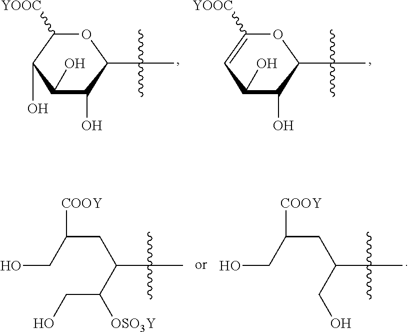

In some embodiments, the compound of Formula (I) is a compound of Formula (Ia)

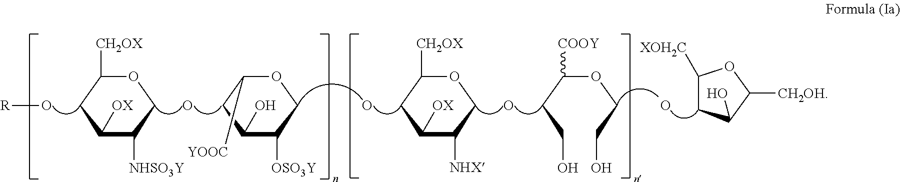

In some embodiments, Y for each occurrence is Na⁺. In some embodiments, R is

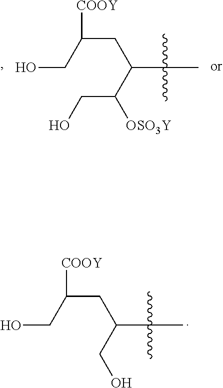

In some embodiments, the LMWH preparation comprises or consists essentially of:

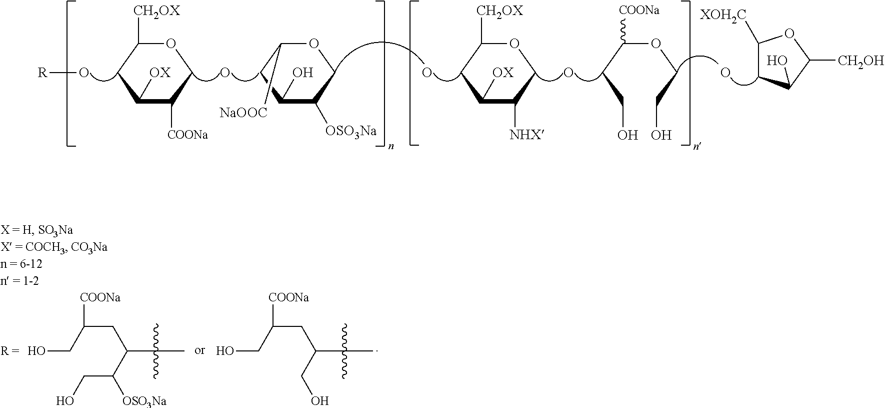

In some embodiments, the LMWH preparation is in liquid form. In some embodiments, formic acid is detectable at a level between 0.0001-1.9% (e.g., 0.001-1.9%, 0.01-1.9%, 0.1-1.9%, 1-1.9%, 0.05-1.9%, 0.5-1.9%, 0.5-1%), e.g., by 1D-NMR. In some embodiments, C₂H₆O₃ is detectable at a level between 0.0001-1.9% (e.g., 0.001-1.9%, 0.01-1.9%, 0.1-1.9%, 1-1.9%, 0.05-1.9%, 0.5-1.9%, 0.5-1%), e.g., by 2D-NMR. In some embodiments, the level of C₂H₆O₃ is determined by 2D-NMR. In some embodiments, the level of formic acid is determined by 1D-NMR technique.

In some embodiments, the LMWH preparation has at least one chain having a glycol split uronic acid residue (U_{G}), wherein formic acid is formic acid is undetectable, e.g., by 1D NMR and/or C₂H₆O₃ is undetectable , e.g., by 2D NMR. In some embodiments, the LMWH preparation has at least one chain having a glycol split uronic acid residue (U_{G}) in the preparation packaged for administration to a human subject, whereinformic acid is detectable in the composition but at less than 2%, 1%, 0.5%, 0.3%, 0.2%, 0.1%, 0.05%, as determined by 1D-NMR and/or C₂H₆O₃ is detectable in the composition but at less than 2%, 1%, 0.5%, 0.3%, 0.2%, 0.1%, 0.05% C₂H₆O₃, as determined by D2-NMR. In some embodiments, the LMWH preparation has at least one chain having a glycol split uronic acid residue (U_{G}) in the preparation packaged for administration to a human subject, wherein formic acid is detectable in the composition at a level between 0.0001-2% (e.g., 0.001-2%, 0.01-2%, 0.1-2%, 1-2%, 0.05-2%, 0.5-2%, 0.5-1%), e.g., NMR (e.g., 1D-NMR, 2D-NMR) and/or C₂H₆O₃ is detectable in the composition at a level between 0.0001-2% (e.g., 0.001-2%, 0.01-2%, 0.1-2%, 1-2%, 0.05-2%, 0.5-2%, 0.5-1%), e.g., NMR (e.g., 1D-NMR, 2D-NMR). In some embodiments, the LMWH preparation has at least one chain having a glycol split uronic acid residue (U_{G}) in the preparation packaged for administration to a human subject, wherein formic acid is wherein formic acid is formic acid is undetectable, e.g., by 1D NMR and/or C₂H₆O₃ is undetectable , e.g., by 2D NMR.

In some embodiments, the packaging is associated with a label, wherein the label indicates a value of the level of formic acid and/or a value of the level of C₂H₆O₃ of the composition. In some embodiments, the label indicates a value of the level of formic acid as determined by an analytical technique, e.g., NMR (e.g., 1D-NMR, D2-NMR) and/or a value of the level of C₂H₆O₃ as determined by an analytical technique, e.g., NMR (e.g., 1D-NMR, D2-NMR) of the composition.

In one aspect, described herein are methods of producing a LMWH preparation having at least one chain having a glycol split uronic acid residue (U_{G}) in the preparation, the method comprising: providing a LMWH preparation having at least one chain having a glycol split uronic acid residue (U_{G}) in the preparation; determining, by performing an analytical technique, e.g., NMR (e.g., ¹H NMR, 1D-NMR, D2-NMR) the presence or absence of a structural signature, (e.g., a structural signature described herein, in the preparation); and based upon the determination of the presence or absence of the structural signature, making a decision whether to process the preparation, (e.g., as described herein), to thereby produce the LMWH preparation.

In one aspect, described herein are methods of a LMWH preparation having at least one chain having a glycol split uronic acid residue (U_{G}) in the preparation, the method comprising: providing a LMWH preparation having at least one chain having a glycol split uronic acid residue (U_{G}) in the preparation; determining, by performing an analytical technique, e.g., NMR (e.g., ¹H NMR, 1D-NMR, D2-NMR) the presence or absence of a structural signature, (e.g., a structural signature described herein, in the preparation); and if the structural signature is present in an amount that indicates that the preparation is stable, formulating the LMWH preparation, e.g., as described herein, to thereby produce the LMWH preparation.

In some embodiments, the LMWH preparation (e.g., the M402 preparation) has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation has the following characteristics: (a) a weight average chain molecular weight between 3,500 and 8,000 Da; (b) anti-Xa activity of less than 20 IU/mg and anti-IIa activity of less than 20 IU/mg; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation is an M402 preparation. In some embodiments, the LMWH preparation is

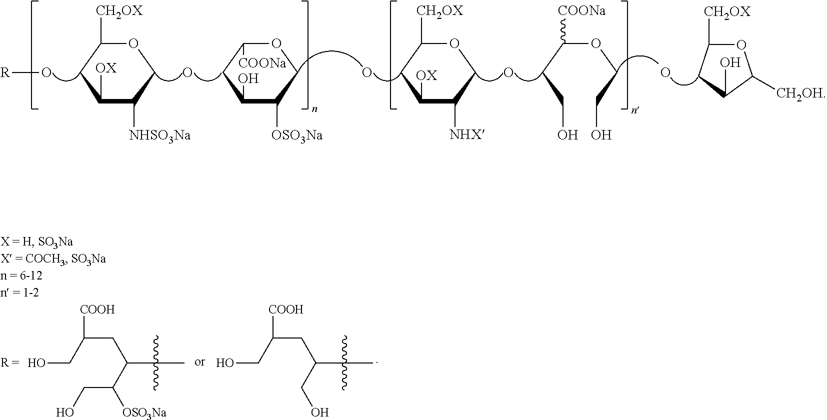

In some embodiments, the analytic technique is a NMR method. In some embodiments, the NMR method is a ¹H NMR method. In some embodiments, method comprises determining by a NMR method, e.g., a ¹H NMR method, whether the preparation is substantially free of formic acid. In some embodiments, if the level of formic acid is above 2%, the preparation is discarded or withheld.

In some embodiments, the method comprises determining by a NMR method, e.g., a ¹H NMR method, whether the preparation is substantially free of:

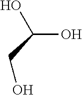

e.g., and if

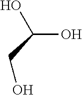

is present in amount of above 2% the preparation, discarding or withholding the preparation.

In one aspect, described herein are methods of screening a LMWH preparation having at least one chain having a glycol split uronic acid residue (U_{G}) in the preparation, the method comprising: providing a LMWH preparation having at least one chain having a glycol split uronic acid residue (U_{G}) in the preparation; determining, by performing an analytical technique, e.g., NMR, the presence or absence of a structural signature, e.g., a structural signature described herein, in the LMWH preparation; and based upon the determination of the presence or absence of the structural signature, making a decision whether to process the preparation, e.g., as described herein, to thereby screen the preparation.

In one aspect, described herein are methods of analyzing a LMWH preparation having at least one chain having a glycol split uronic acid residue (U_{G}) in the preparation, the method comprising: providing a LMWH preparation having at least one chain having a glycol split uronic acid residue (U_{G}) in the preparation; determining, by performing an analytical technique, e.g., NMR, the presence or absence of a structural signature, e.g., a structural signature described herein, in the preparation; and based upon the determination of the presence or absence of the structural signature, making a decision whether to process the preparation, e.g., as described herein, to thereby analyze the preparation.

In one aspect, described herein are methods for manufacturing M402, the method comprising: obtaining a first polysaccharide preparation comprising unfractionated heparin; depolymerizing the first polysaccharide preparation for a time and under conditions to obtain a second polysaccharide preparation having a weight average molecular weight of 3000-8000 Da; modifying the second polysaccharide preparation to obtain a third polysaccharide preparation comprising at least one chain having a glycol split uronic acid residue, formulating and/or releasing the third polysaccharide preparation for administration to a human subject if formic acid is detectable in the composition but at less than 2%, 1%, 0.5%, 0.3%, 0.2%, 0.1%, 0.05%, 1D-NMR and/or C₂H₆O₃ is detectable in the composition but at less than 2%, 1%, 0.5%, 0.3%, 0.2%, 0.1%, 0.05%, as determined by 2D-NMR, to thereby manufacture M402.

In some embodiments, the first polysaccharide preparation is depolymerized using nitrous acid based depolymerization. In some embodiments, the second polysaccharide preparation is modified by subjecting the second polysaccharide preparation periodate oxidation. In some embodiments the method further comprises purifying the third polysaccharide preparation, e.g., by a chromatographic method (e.g. gel filtration chromatography). In some embodiments the method further comprises reducing the third polysaccharide preparation, e.g., by treatment with a reducing agent (e.g., sodium borohydride).

In one aspect, described herein are methods for manufacturing M402, the method comprising: obtaining a first polysaccharide preparation comprising unfractionated heparin; depolymerizing the first polysaccharide preparation for a time and under conditions to obtain a second polysaccharide preparation having a weight average molecular weight of 3000-8000 Da; modifying the second polysaccharide preparation to obtain a third polysaccharide preparation comprising at least one chain having a glycol split uronic acid residue; formulating and/or releasing the third polysaccharide preparation for administration to a human subject if formic acid is undetectable in the composition as determined by 1D-NMR and/or C₂H₆O₃ is undetectable in the composition as determined by 2D-NMR; to thereby manufacture M402.

In some embodiments, the first polysaccharide preparation is depolymerized using nitrous acid based depolymerization. In some embodiments, the second polysaccharide preparation is modified by subjecting the second polysaccharide preparation periodate oxidation. In some embodiments the method further comprises purifying the third polysaccharide preparation, e.g., by a chromatographic method (e.g. gel filtration chromatography). In some embodiments the method further comprises reducing the third polysaccharide preparation, e.g., by treatment with a reducing agent (e.g., sodium borohydride).

In one aspect, described herein are methods for manufacturing a an M402 preparation, the method comprising: (a) obtaining a preparation of unfractionated heparin (UFH); (b) depolymerizing the UFH for a time and under conditions to obtain a first polysaccharide preparation having a weight average molecular weight of 3000-8000 Da; (c) glycol splitting the first polysaccharide preparation to obtain a second polysaccharide preparation; (d) reducing and purifying the second polysaccharide preparation; (e) measuring formic acid and C₂H₆O₃ in the reduced and purified second polysaccharide preparation; and (f) processing the second polysaccharide preparation as M402 drug product or drug substance if formic acid is less than 2%, 1%, 0.5%, 0.3%, 0.2%, 0.1%, 0.05% as determined by 1D NMR and C₂H₆O₃ is less than 2%, 1%, 0.5%, 0.3%, 0.2%, 0.1%, 0.05% as determined by 2D NMR; to thereby manufacture a M402 preparation.

In some embodiments, the first polysaccharide preparation is depolymerized using nitrous acid based depolymerization. In some embodiments, the second polysaccharide preparation is modified by subjecting the second polysaccharide preparation periodate oxidation. In some embodiments the method further comprises purifying the third polysaccharide preparation, e.g., by a chromatographic method (e.g. gel filtration chromatography). In some embodiments the method further comprises reducing the third polysaccharide preparation, e.g., by treatment with a reducing agent (e.g., sodium borohydride).

In one aspect, described herein are methods for manufacturing a M402 preparation, the method comprising: (a) obtaining a preparation of unfractionated heparin (UFH); (b) depolymerizing the UFH for a time and under conditions to obtain a first polysaccharide preparation having a weight average molecular weight of 3000-8000 Da; (c) glycol splitting the first polysaccharide preparation to obtain a second LMWH preparation; (d) reducing and purifying the second polysaccharide preparation; (e) measuring formic acid and C₂H₆O₃ in the reduced and purified second polysaccharide preparation; and (f) processing the second polysaccharide preparation as M402 drug product or drug substance if formic acid is less than 2%, 1%, 0.5%, 0.3%, 0.2%, 0.1%, 0.05% as determined by 1D NMR, to thereby manufacture a M402 preparation.

In some embodiments, the first polysaccharide preparation is depolymerized using nitrous acid based depolymerization. In some embodiments, the second polysaccharide preparation is modified by subjecting the second polysaccharide preparation periodate oxidation. In some embodiments the method further comprises purifying the third polysaccharide preparation, e.g., by a chromatographic method (e.g. gel filtration chromatography). In some embodiments the method further comprises reducing the third polysaccharide preparation, e.g., by treatment with a reducing agent (e.g., sodium borohydride).

In one aspect, described herein are methods for manufacturing a M402 preparation, the method comprising: (a) obtaining a preparation of unfractionated heparin (UFH); (b) depolymerizing the UFH for a time and under conditions to obtain a first polysaccharide preparation having a weight average molecular weight of 3000-8000 Da; (c) glycol splitting the first polysaccharide preparation to obtain a second polysaccharide preparation; (d) reducing and purifying the second polysaccharide preparation; (e) measuring formic acid and C₂H₆O₃ in the reduced and purified second polysaccharide preparation; and (f) processing the second polysaccharide preparation as M402 drug product or drug substance if C₂H₆O₃ is less than 2%, 1%, 0.5%, 0.3%, 0.2%, 0.1%, 0.05% as determined by 2D NMR, to thereby manufacture a M402 preparation.

In some embodiments, the first polysaccharide preparation is depolymerized using nitrous acid based depolymerization. In some embodiments, the second polysaccharide preparation is modified by subjecting the second polysaccharide preparation periodate oxidation. In some embodiments the method further comprises purifying the third polysaccharide preparation, e.g., by a chromatographic method (e.g. gel filtration chromatography). In some embodiments the method further comprises reducing the third polysaccharide preparation, e.g., by treatment with a reducing agent (e.g., sodium borohydride).

Compositions described herein include, inter alia, pharmaceutical compositions comprising a LMWH preparation having at least one chain having a glycol split uronic acid residue (U_{G}) in the preparation (e.g., a LMWH preparation described herein, e.g., an M402 preparation). In some embodiments, the composition is substantially free of formic acid (e.g., less than 1%, 0.5%, 0.1%, 0.05%, 0.02%, 0.01%, 0.001% weight/weight (w/w) formic acid as determined by 1D-NMR) and/or C₂H₆O₃ (e.g., less than 1%, 0.5%, 0.1%, 0.05%, 0.02%, 0.01%, 0.001% (w/w) C₂H₆O₃, as determined by 2D-NMR). In some embodiments, the LMWH preparation contains less than, e.g., 1%, 0.5%, 0.1%, 0.02%, 0.01%, 0.001% (w/w) of formic acid and/or, when stored at about 4° C. (e.g., 2-6° C., 3-4° C., 3-6° C., 4-5° C., 4-6° C.) for 3 months or more, (e.g., 4, 5, 6, 7, 8, 9, 10, 11, 12, 24 months or more).

In some embodiments, formic acid is undetectedable in the LMWH preparationusing 1D-NMR. In some embodiments, formic acid is detectable in the LMWH preparation at a level between 0.0001-1% (e.g., 0.001-1%, 0.01-1%, 0.1-1%, 1-1%, 0.05-1%, 0.5-1%, 0.5-1%) as determined by 1D-NMR. In some embodiments, C₂H₆O₃ is undetectedable in the LMWH preparationusing 2D-NMR. In some embodiments, C₂H₆O₃ is detectable in the LMWH preparation at a level between 0.0001-1% (e.g., 0.001-1%, 0.01-1%, 0.1-1%, 1-1%, 0.05-1%, 0.5-1%, 0.5-1%) as determined by 2D-NMR. In some embodiments, the level of C₂H₆O₃ in the LMWH preparation is determined by an NMR technique (e.g., 1D-NMR, 2D-NMR). In some embodiments, the level of formic acid in the LMWH preparation is determined by an NMR technique (e.g., 1D-NMR, 2D-NMR).

In some embodiments, the LMWH preparation has the following characteristics:

(a) a weight average chain molecular weight between 3,500 and 8,000 Da, (e.g., 5,000-8,000 Da); (b) anti-Xa activity of less than 50 IU/mg (e.g., less than 40, 30, 20, 15, 10, 5, 2, 1, 0.5, 0.1, 0.01, 0.001 IU/mg) and anti-lla activity of less than 50 IU/mg (e.g., less than 40, 30, 20, 15, 10, 5, 2, 1, 0.5, 0.1, 0.01, 0.001 IU/mg); (c) greater than 5% (e.g., 10, 20) and less than 50% (e.g., less than 40, 30, 25, 20, 10) glycol split uronic acid residues; (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da; and (e) substantially free of formic acid (e.g., less than 2%, 1%, 0.5%, 0.3%, 0.2%, 0.1%, 0.05% formic acid, as determined by 1D-NMR) and/or C₂H₆O₃ (e.g., less than 2%, 1%, 0.5%, 0.3%, 0.2%, 0.1%, 0.05% C₂H₆O₃, as determined by 2D-NMR.

In some embodiments, the LMWH preparation is an M402 preparation. In some embodiments, the M402 preparation is Necuparinol. In some embodiments, the M402 preparation the preparation identified as M402 in Zhou et al., M402, a Novel Heparan Sulfate Mimetic, Targets Multiple Pathways Implicated in Tumor Progression and Metastasis, PLoS ONE 6(6): e21106. Doi:10.1371/journal.pone.0021106, (Jun. 16, 2011). In some embodiments, the LMWH preparation comprises or consists essentially of a polysaccharide of Formula (I)

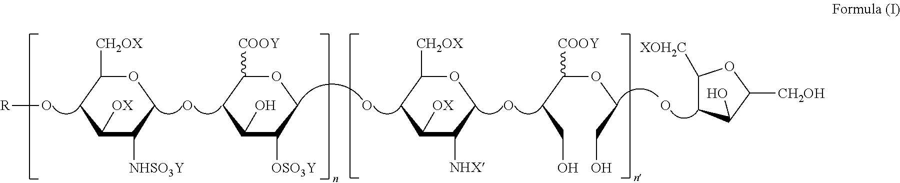

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and
R is

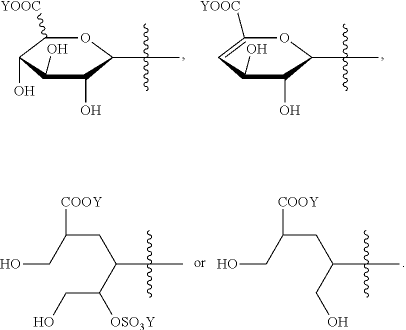

In some embodiments, the compound of Formula (I) is a compound of Formula (Ia)

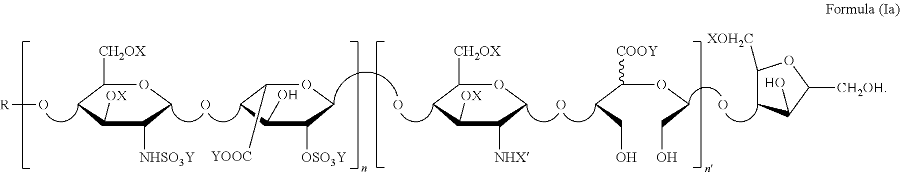

In some embodiments, Y for each occurrence is Na⁺.

In some embodiments, R is

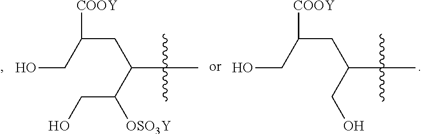

In some embodiments, the LMWH preparation comprises or consists essentially of:

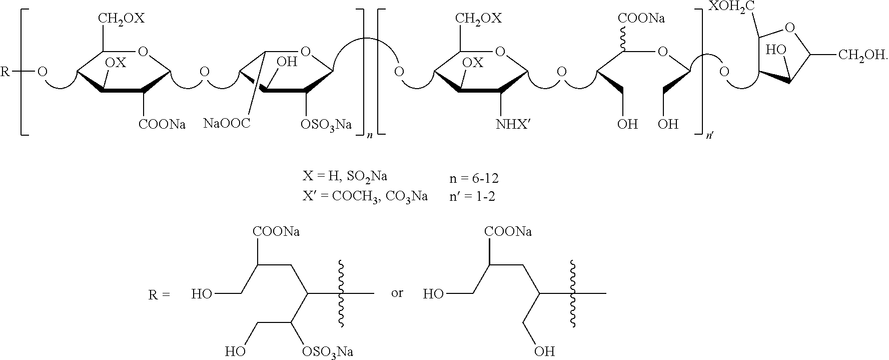

which is also represented as:

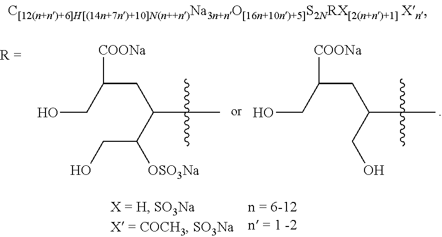

Methods disclosed herein are useful, inter alia, in analyzing a LMWH preparation having at least one chain having a glycol split uronic acid residue (U_{G}) in the preparation, (e.g., a low molecular weight heparin (LMWH) preparation described herein, e.g., an M402 preparation), to determine whether the preparation meets one or both of the structural signatures provided herein and using said determination to accept or reject a batch of a LMWH preparation having at least one chain having a glycol split uronic acid residue (U_{G}) in the preparation (e.g., a LMWH preparation described herein, e.g., an M402 preparation). For example, the disclosure provides methods for analyzing a LMWH preparation having at least one chain having a glycol split uronic acid residue (U_{G}) in the preparation to determine whether the preparation meets one or both of the structural signatures provided herein and using said determination to accept the preparation as M402. The disclosure also features, inter alia, methods of producing or manufacturing a LMWH preparation comprising: providing a LMWH preparation having at least one chain having a glycol split uronic acid residue (U_{G}) in the preparation (e.g., a LMWH preparation described herein, e.g., an M402 preparation), and receiving (e.g., acquiring) a value for the absence, presence, or level of one or both of the structural signatures disclosed herein in the preparation (e.g., a value determined by performing an analytical technique (e.g., NMR)), determining whether said value meets a value disclosed herein for one or both of the structural signatures disclosed herein and, optionally, based upon the determination, further processing of the preparation (e.g., withholding, releasing, formulating the preparation), to thereby produce a LMWH preparation. For example, the disclosure provides methods for producing or manufacturing a LMWH having at least one chain having a glycol split uronic acid residue (U_{G}) in the preparation comprising: obtaining a preparation of unfractionated heparin, depolymerizing the UFH under conditions and for a time sufficient to obtain a first LMWH preparation having a weight average molecular weight of 3000-8000 Da, glycol splitting the first preparation to obtain a second preparation having at least one chain having a glycol split uronic acid residue (U_{G}) in the preparation, measuring one or both of formic acid and/or C₂H₆O₃ in the second LMWH preparation, determining whether the one or both of formic acid and/or C₂H₆O₃ measured meets a value disclosed herein for the one or both of formic acid and/or C₂H₆O₃, and using the determination to release the second LMWH preparation for further processing. For example, the disclosure provides methods for producing or manufacturing a LMWH having at least one chain having a glycol split uronic acid residue (U_{G}) in the preparation comprising: obtaining a preparation of unfractionated heparin, depolymerizing the UFH under conditions and for a time sufficient to obtain a first LMWH preparation having a weight average molecular weight of 3000-8000 Da, glycol splitting the first preparation to obtain a second preparation having at least one chain having a glycol split uronic acid residue (U_{G}) in the preparation, reducing and purifying the second LMWH preparation to obtain a third LMWH preparation, measuring one or both of formic acid and/or C₂H₆O₃ in the third LMWH preparation, determining whether the one or both of formic acid and/or C₂H₆O₃ measured meets a value disclosed herein for the one or both of formic acid and/or C₂H₆O₃, and using the determination to release the third LMWH preparation as M402 (e.g., for further processing to obtain M402 drug substance or M402 drug product).

The disclosure also features, inter alia, methods of producing a LMWH preparation comprising: receiving a LMWH preparation having at least one chain having a glycol split uronic acid residue (U_{G}) in the preparation (e.g., a LMWH preparation described herein, e.g., an M402 preparation), and determining a value for the absence, presence, or level of a structural signature disclosed herein in the preparation (e.g., a value for the absence, presence and/or amount of a structural signature in the LMWH preparation determined by performing an analytical technique (e.g., NMR), and, optionally, based upon the determination, further processing of the preparation (e.g., withholding, releasing, formulating the preparation) or providing instruction to further process the preparation, e.g., to another party, to thereby produce a LMWH preparation.

The disclosure also features, inter alia, methods of producing a LMWH preparation comprising: producing a LMWH preparation having at least one chain having a glycol split uronic acid residue (U_{G}) in the preparation (e.g., a LMWH preparation described herein, e.g., an M402 preparation), and processing the LMWH preparation if a value for the absence, presence or amount of a structurally signature in the preparation (e.g., a value for the absence, presence and/or amount of a structural signature in the LMWH preparation determined by performing an analytical technique (e.g., NMR), meets a predetermined value. The processing can include, e.g., formulating the preparation, packaging the preparation, portioning into smaller aliquots, disposing into a container, e.g., a gas or liquid container, or associating the preparation with a label. The methods disclosed herein also feature methods of evaluating a LMWH preparation having at least one chain having a glycol split uronic acid residue (U_{G}) in the preparation, e.g., as described herein, by an analytical technique described herein, for the absence, presence and/or amount of a structural signature described herein.

In certain embodiments, the LMWH preparation is a drug substance and, e.g., the processing comprises one or more of formulating; processing into a drug product; combining with a second component, e.g., an excipient or buffer, depending upon the determination. In one embodiment, the LMWH preparation is drug product and, e.g., the processing comprises one or more of: portioning into smaller or larger aliquots; disposing into a container, e.g., a gas or liquid tight container; packaging; associating with a label; shipping or moving to a different location, depending on the determination. In one embodiment, the drug product is a pharmaceutical composition described herein, e.g., a pharmaceutical composition comprises an M402 preparation described herein.

In certain embodiments, the LMWH preparation lacks substantial anticoagulant activity (e.g., the preparation of polysaccharides has reduced anticoagulant activity but retains an anti-proliferative activity (e.g., anti-metastatic, anti-angiogenic, anti-fibrotic and/or anti-inflammatory property). For example, the LMWH preparation can have an anti-Xa activity of less than 30 IU/mg, 20 IU/mg, 15 IU/mg, 10 IU/mg, 5 IU/mg, 3 IU/mg, 2 IU/mg or less and/or an anti-IIa activity of less than 30 IU/mg, 20 IU/mg, 15 IU/mg, 10 IU/mg, 5 IU/mg, 3 IU/mg, 2 IU/mg, 1 IU/mg or less. In certain embodiments, the LMWH preparation is an M402 preparation described herein.

In some embodiments, the LMWH preparation described herein is included in a pharmaceutical composition (e.g., a drug product). In certain embodiments, the LMWH preparation is present in the pharmaceutical composition in an amount of about 150 mg mL⁻¹. In some embodiments, the LMWH preparation described herein is present in the composition in an amount less than 150 mg mL⁻¹. In some embodiments, the LMWH preparation described herein is present in the pharmaceutical composition in an amount greater than 150 mg mL⁻¹. In some embodiments, the LMWH preparation is M402, and M402 is present in the pharmaceutical composition in an amount between 100-500 mg mL⁻¹, e.g., 1, 10, 50, 75, 100, 125, 175, 200, 225, 250, 275, 300, 325, 350, 375, 400, 425, 450, or 500 mg mL⁻¹. In some embodiments, the LMWH preparation described herein is present in the pharmaceutical composition in an amount greater than 1 mg mL⁻¹.

In preferred embodiments, when the method includes a processing step, the processing comprises one or more of: processing into a drug product, e.g., formulating, combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a drug product, shipping, moving to a different location, formulating, labeling, packaging, releasing into commerce, or selling or offering for sale, depending on the determination.

In some aspects, the disclosure features a method of manufacturing a LMWH preparation having at least one chain having a glycol split uronic acid residue (U_{G}) in the preparation (e.g., a LMWH preparation described herein, e.g., an M402 preparation), the method comprising:

producing a LMWH preparation described herein, e.g., by a method described herein;

receiving (or acquiring) a value for one or both structural signatures disclosed herein in the LMWH preparation, wherein, e.g., wherein the one or both structural signatures is indicative of stability of the LMWH preparation, and

processing the LMWH preparation if the value meets a predetermined value for one or more structural signatures described herein, e.g., stability, thereby manufacturing the LMWH preparation.

In one embodiment, the production of the LMWH preparation comprises formulating the LMWH preparation into a pharmaceutical composition, e.g., adding one or more excipient or buffer, and the value received or acquired is for the pharmaceutical composition comprising the LMWH preparation. In embodiments, the processing can comprise, e.g., one or more of: portioning into smaller or larger aliquots; disposing into a container, e.g., a gas or liquid tight container; packaging; associating with a label; shipping or moving to a different location. In one embodiment, the preparation is formulated, e.g., as described in U.S. Provisional Application No.: 61/828,116 entitled Pharmaceutical Compositions, filed May 28, 2013, Inventors: Kelly Neelon, Gary Mills, and James Anderson, which application and/or methods are hereby incorporated by reference.

In some embodiments, a value or predetermined value, for one or both of the signatures disclosed herein, is a release specification for the LMWH preparation.

In one embodiment, the predetermined value for a structural signature is a function of the range of values for that signature observed for multiple samples or batches of a target LMWH preparation, e.g., commercially available samples or batches of a LMWH preparation. In one embodiment, the predetermined value is a numerical value such as a single number, or a range.

In some embodiments, the predetermined value is recorded in a quality specification for the LMWH preparation, e.g., a certificate of analysis (COA), certificate of testing (COT), Master Batch Record (MBR).

In preferred embodiments, the processing comprises one or more of: processing into a drug product, e.g., formulating, combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a drug product, shipping, moving to a different location, formulating, labeling, packaging, releasing into commerce, or selling or offering for sale, depending on the determination.

In certain embodiments, the LMWH preparation is a drug substance and, e.g., the processing comprises one or more of formulating; processing into a drug product; combining with a second component, e.g., an excipient or buffer, depending upon the determination. In one embodiment, the LMWH preparation is drug product and, e.g., the processing comprises one or more of: portioning into smaller or larger aliquots; disposing into a container, e.g., a gas or liquid tight container; packaging; associating with a label; shipping or moving to a different location, depending on the determination. In one embodiment, the drug product is a pharmaceutical composition described herein, e.g., a pharmaceutical composition comprises an M402 preparation described herein.

In some embodiments of the aspects described herein, the structural signature is indicative of the stability of the LMWH preparation, e.g., of the LMWH preparation in a pharmaceutical composition. Exemplary structural signatures include, but are not limited to, formic acid and/or

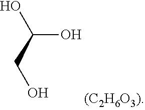

In one embodiment, when a LMWH preparation is substantially free of formic acid and/or

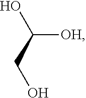

the LMWH preparation is a stable preparation. In preferred embodiments, the LMWH preparation contains less than, e.g., 1%, 0.5%, 0.1%, 0.05%, 0.02%, 0.01%, 0.001% weight/weight (w/w) of formic acid as determined by 1D NMR and/or

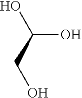

as determined by 2D NMR, when stored at

4° C. (e.g., 2-6° C., 3-4° C., 3-6° C., 4-5° C., 4-6° C.) for 3 months or more, (e.g., 4, 5, 6, 7, 8, 9, 10, 11, 12, 24 months or more).

### Nuclear Magnetic Resonance (NMR)

In certain embodiments, the analytical technique performed is a NMR method (e.g., 1D NMR, e.g., ¹H NMR). In a preferred embodiment, the analytical technique performed is 1D NMR.

In one embodiment, the NMR method is heteronuclear single quantum coherence spectroscopy (HSQC). In some instances, methods for NMR (e.g., 1D and/or 2D NMR) are known in the art and/or are disclosed, for example, in literature publications such as Guerrini et al, Semin. Thromb. Hemos., 33(5):478-87 (2007) and Guerrini et al., Anal. Biochem., 1:337(1):35-47 (2005).

In an embodiment, the method further comprises determining by a NMR method, e.g.,¹D NMR or HSQC, if the LMWH preparation contains formic acid. In an embodiment, if formic acid is not detected (e.g., above background) or the LMWH preparation is substantially free of formic acid, as detected by a NMR method, e.g., a ¹H NMR method or a HSQC method, the preparation is formulated; processed into a drug product; combined with a second component, e.g., an excipient or buffer; portioned into smaller or larger aliquots; disposed into a container, e.g., a gas or liquid tight container; packaged; associated with a label; shipped or moved to a different location. In an embodiment, if the LMWH preparation contains formic acid or is not substantially free of formic acid, as detected by a NMR method, e.g., a ¹H NMR method or a HSQC method, the preparation is discarded or withheld.

The term “substantially free of formic acid” as used herein refers to a composition that has 1% by weight formic acid or less, e.g., less than 0.5%, 0.1%, 0.05%, 0.02%, 0.01%, 0.001% weight/weight (w/w) as determined by 1D NMRwhen stored at 4° C. for 3 months, as measured by an analtyical method, e.g., an NMR method, e.g., a 1D NMR method, e.g., a ¹H NMR method.

In an embodiment, if the preparation contains 0.05% by weight formic acid or above (e.g., 0.05%, 0.1%, 0.3, 0.5%, 1%, 2%, 3%), as determined by a 1D NMR method, e.g., a ¹H NMR method, or a HSQC method, the preparation is discarded, reprocessed, or withheld. In an embodiment, if the preparation contains 0.1% by weight formic acid or above, as determined by a 1D NMR method, e.g., a ¹H NMR method, or a HSQC method, the preparation is discarded or withheld. In an embodiment, if the preparation contains 0.3% by weight formic acid or above, as determined by a 1D NMR method, e.g., a ¹H NMR method, or a HSQC method, the preparation is discarded or withheld. In an embodiment, if the preparation contains 0.5% by weight formic acid or above, as determined by a 1D NMR method, e.g., a ¹H NMR method, or a HSQC method, the preparation is discarded or withheld. In an embodiment, if the preparation contains 1% by weight formic acid or above, as determined by a 1D NMR method, e.g., a ¹H NMR method, or a HSQC method, the preparation is discarded or withheld. In an embodiment, if the preparation contains 2% by weight formic acid or above, as determined by a 1D NMR method, e.g., a ¹H NMR method, or a HSQC method, the preparation is discarded or withheld. In an embodiment, if the preparation contains 3% by weight formic acid or above, as determined by a 1D NMR method, e.g., a ¹H NMR method, or a HSQC method, the preparation is discarded or withheld.

In an embodiment, if the level of formic acid in the preparation is increased (e.g., by a statistically significant amount) compared to a reference value as described herein (e.g., a value provided by a reference standard, e.g., a preparation of the polysaccharide maintained under standard conditions for a predetermined period of time, e.g., stored at 4° C. for 3 months) as measured by an analtyical method, e.g., a NMR method, e.g., a 1D NMR method, e.g., a ¹H NMR method, or a HSQC method, the preparation is discarded or withdrawn. In an embodiment, if the level of formic acid in the preparation is the same or similar to a reference value as described herein (e.g., a value provided by a reference standard, e.g., a preparation of the polysaccharide maintained under standard conditions for a predetermined period of time, e.g., stored at 4° C. for 3 months) as determined by a 1D NMR method, e.g., a ¹H NMR method, or a HSQC method, the preparation is formulated; processed into a drug product; combined with a second component, e.g., an excipient or buffer; portioned into smaller or larger aliquots; disposed into a container, e.g., a gas or liquid tight container; packaged; associated with a label; shipped or moved to a different location.

In an embodiment, the method comprises determining by a 1D NMR method or a HSQC method, if the LMWH preparation contains:

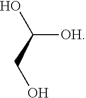

In a preferred embodiment, if the LMWH preparation is substantially free of or contains less than 1%, 0.5%, 0.3%, 0.1%, or 0.05%:

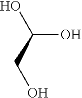

by weight, as determined by a 1D NMR method or a HSQC method, the preparation is formulated; processed into a drug product; combined with a second component, e.g., an excipient or buffer; portioned into smaller or larger aliquots; disposed into a container, e.g., a gas or liquid tight container; packaged; associated with a label; shipped or moved to a different location.

In a preferred embodiment, if the LMWH preparation is not substantially free of or contains:

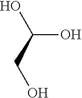

above 0.05% (e.g., above 0.1%, above 0.3, above 0.5%, above 1%, above 2%, above 3%) by weight, as determined by a 1D NMR method or a HSQC method, the preparation is discarded or withheld.

A person of ordinary skill in the art will appreciate that assessment of stability need not be expressed using the units or metrics disclosed herein. Nevertheless, comparison of stability will be conducted using the methods, units, and/or metrics disclosed herein. More specifically, an assessed stability is equivalent to those disclosed herein as long as the assessed stability meets the herein disclosed stability when the herein disclosed methods, units and/or metrics are used, e.g., allowing for the sensitivity (e.g., analytical variability) of the method being used to measure the value.

### BRIEF DESCRIPTION OF THE FIGURES

FIG. 1 shows a 1D ¹H-NMR spectra of M402 samples stored at 4° C. (top) and 40° C./75% RH (bottom) for three and six months. Signals due to degraded material are indicated with circles.

### DETAILED DESCRIPTION OF THE INVENTION

The disclosure is based, in part, on the discovery of structural signatures that are associated with LMWH preparations having at least one chain having a glycol split uronic acid residue (U_{G}) (e.g., a low molecular weight heparin (LMWH) preparation described herein, e.g., an M402 preparation). These structures can be used, e.g., to analyze or evaluate such LMWH preparations to determine, inter alia, the stability of the preparation. The analysis or evaluation can be useful to determine production parameters, determine batch variability, determine formulation conditions and/or storage conditions, determine compliance with drug specifications (e.g., a release standard or production standard), etc.

A “stable” LMWH preparation, as used herein, is a preparation that is substantially free from a degradation product. “Substantially free from a degradation product” means that the preparation contains less than 1%, 0.5%, 0.1%, 0.05%, 0.02%, 0.01%, 0.001% weight/weight (w/w) formic acid as determined by 1D-NMR and/or less than 1%, 0.5%, 0.1%, 0.05%, 0.02%, 0.01%, 0.001% (w/w) C₂H₆O₃, as determined by 2D-NMR.

As used herein, a “batch” of a LMWH preparation refers to a single production run of the LMWH. Evaluation of different batches thus means evaluation of different production runs or batches. As used herein “sample(s)” refer to separately procured samples. For example, evaluation of separate samples could mean evaluation of different commercially available containers or vials of the same batch or from different batches.

As used herein, “acquire” or “acquiring” refers to obtaining possession of a physical entity, or a value, e.g., a numerical value, by “directly acquiring” or “indirectly acquiring” the physical entity or value. “Directly acquiring” means performing a process (e.g., performing an assay or test on a sample or “analyzing a sample” as that term is defined herein) to obtain the physical entity or value. “Indirectly acquiring” refers to receiving the physical entity or value from another party or source (e.g., a third party laboratory that directly acquired the physical entity or value). Directly acquiring a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, analyzing a sample includes performing a process that involves a physical change in a sample or another substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Analyzing a sample can include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

A “buffer” as used herein is an agent that maintains a stable pH in a solution within a specific pH range. Buffering ranges are determined by pKa. An aqueous formulation can be prepared including the therapeutic agent in a pH-buffered solution.

### Reference Standards and Reference Values

A “reference value” is a set value for the presence of a structural signature herein in a preparation. The reference value can be numerical or non-numerical. The reference value can also be a value or a plurality of values for the presence of more than one structural signature in a preparation. The reference value can be a qualitative value, e.g., a relative increase or decrease; a yes or no; present or not present; present or not present at a preselected level of detection, or graphic or pictorial. For example, the reference value can correspond to a peak in an NMR spectrum corresponding to a structure present or not present in the preparation when analyzed by NMR, e.g., a 1D NMR method described herein. The reference value can also be a release standard (a release standard is a standard which should be met to allow commercial sale of a product) or production standard. The reference value can be a statistical function, e.g., an average, of a number of values. The reference value can be a function of another value, e.g., of the presence or distribution of a second entity present in the sample, e.g., an internal standard. The reference value can be any value or range disclosed herein.

### LMWH Preparations

The LMWH preparations described herein can be a low molecular weight heparin (LMWH) preparation having at least one chain having a glycol split uronic acid residue (U_{G}) in the preparation.

The disclosed LMWH preparations can be designed to lack substantial anticoagulant activity while retaining clinically advantageous properties. Properties of the LMWH preparations include, e.g., lacking substantial anticoagulant activity, e.g., anti-IIa activity less than 50 IU/mg, anti-Xa activity less than 50 IU/mg, and having anti-metastatic, anti-angiogenic, anti-fibrotic and/or anti-inflammatory activity.

In some embodiments, the LMWH preparation comprises at least one chain having a glycol split uronic acid residue (U_{G}) and, e.g., the preparation can lack substantial anticoagulant activity (e.g., preparations of polysaccharides that have reduced anticoagulant activity) but retain activity in other non-coagulation mediated biological processes. For example, these LMWH preparations can have one or more of the following features: 1) anti-Xa activity, e.g., less than 50 IU/mg, 20 IU/mg, 10 IU/mg, 5 IU/mg, 3 IU/mg, 2 IU/mg, 1 IU/mg or less, and 2) anti-metastatic, anti-angiogenic, anti-fibrotic and/or anti-inflammatory activity. A LMWH preparation provided herein can also have one or more of the following characteristics: the preparation has glycol split uronic acid residues (U_{G}) (e.g., less than 50%, 40%, 30%, 20% glycol split uronic acid residues (U_{G})); the preparation has no more than 3 glycol split uronic acid residues (U_{G}) per polysaccharide chain; the preparation has greater than 40% U_{2S}H_{NS,6S} disaccharide residues present in the chains of the preparation; the degree of desulfation of the preparation is less than 40%; one or more polysaccharide chains in the preparation have a 2,5-anhydromannitol residue at the reducing end. In some preferred embodiments, the weight average molecular weight of the preparation is between 3,500 and 8,000 Da, e.g., between 4,000 and 8,000 Da; and a molecular weight distribution such that 10-50% (e.g., 10-40%, 10-30%, 15-30% or 15-25%) of the oligosaccharides of the preparation have a molecular weight <3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight >8000 Da.

In some embodiments, the LMWH preparation has a weight average molecular weight between 6,000 and 15,000 Da, e.g., between 10,000 and 14,000 Da. In other embodiments, the preparation has a weight average molecular weight between 3,000 and 8,000 Da.

Certain embodiments include a LMWH preparation having the following characteristics: (a) a weight average molecular weight between 3,500 and 8,000 Da, e.g., a weight average molecular weight described herein; (b) anti-Xa activity and/or anti-IIa activity, e.g., less than 50 IU/mg (e.g., anti-Xa activity less than about 40 IU/mg, 30 IU/mg, 20 IU/mg, 15 IU/mg, 10 IU/mg, 5 IU/mg, 4 IU/mg, 3 IU/mg, 2 IU/mg, or 1 IU/mg, and anti-IIa activity less than about 40 IU/mg, 30 IU/mg, 20 IU/mg, 10 IU/mg, 5 IU/mg, 4 IU/mg, 3 IU/mg, 2 IU/mg, or 1 IU/mg); and (c) less than 50% glycol split uronic acid residues (e.g., less than 40%, 30%, 25%, or 20% glycol split uronic acid residues but more than 1%, 5%, 10%, 15%) in the preparation. In some embodiments, the preparation contains between 5% and 50% glycol split uronic acid residues (e.g., between 5% and 40%, 5% and 30%, 10% and 50%, 10% and 40%, 10% and 30%, or 10 and 20% glycol split uronic acid residues). Preferably, the preparation has a molecular weight distribution such that 10-50% (e.g., 10-40%, 10-30%, 15-30% or 15-25%) of the oligosaccharides of the preparation have a molecular weight <3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight >8000 Da.

Certain embodiments include a LMWH preparation having the following characteristics: (a) a weight average chain molecular weight between 3,500 and 8,000 Da; (b) anti-Xa activity of less than 20 IU/mg and anti-Ha activity of less than 20 IU/mg; and (c) greater than 5% and less than 25%, e.g., less than 20, less than 10, glycol split uronic acid residues. Certain embodiments include a LMWH preparation having the following characteristics: (a) a weight average chain molecular weight between 3,500 and 7,000 Da; (b) anti-Xa activity of less than 20 IU/mg and anti-Ha activity of less than 20 IU/mg; and (c) greater than 5% and less than 20% glycol split uronic acid residues.

Certain embodiments include a LMWH preparation having the following characteristics: (a) a weight average chain molecular weight between 3,500 and 8,000 Da; (b) anti-Xa activity of less than 20 IU/mg and anti-Ha activity of less than 20 IU/mg; and (c) greater than 5% and less than 25%, e.g., less than 20, less than 10, glycol split uronic acid residues; wherein the preparation has polysaccharide chains of the preparation having greater than 40%, e.g., greater than 50%, 60%,70%, U_{2S}H_{NS,6S} disaccharide residues.

In some embodiments, the LMWH preparation has one or more chains having a glycol split uronic acid residue and each polysaccharide chain of the preparation having no more than 3, e.g., no more than 2, no more than 1, glycol split uronic acid residues (U_{G}). In some embodiments, the LMWH preparation has one or more chain having a glycol split uronic acid residue and each polysaccharide chain of the preparation having no more than 2 glycol split uronic acid residues (U_{G}). In some embodiments, the LMWH preparation has one or more chains having a glycol split uronic acid residue and each polysaccharide chain of the preparation having no more than 1 glycol split uronic acid residues (U_{G}).

In some embodiments, the LMWH preparation preparation has the following characteristics: (a) a weight average chain molecular weight between 3,500 and 8,000 Da; (b) anti-Xa activity of less than 20 IU/mg and anti-Ha activity of 1 IU/mg or less; (c) greater than 5% and less than 25% glycol split uronic acid residues; and (d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

In some embodiments, the LMWH preparation comprises a polysaccharide of Formula (I)

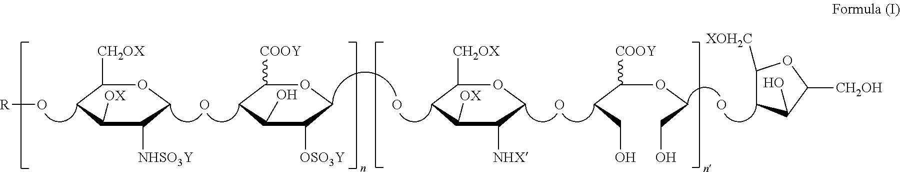

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and
R is

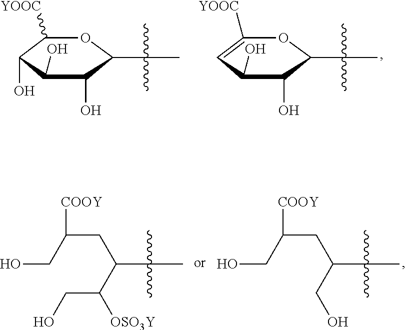

each Y is independently a singularly charged cation such as Na⁺, K⁺, or NH₄⁺.

In some embodiments, the compound of Formula (I) is a compound of Formula (Ia)

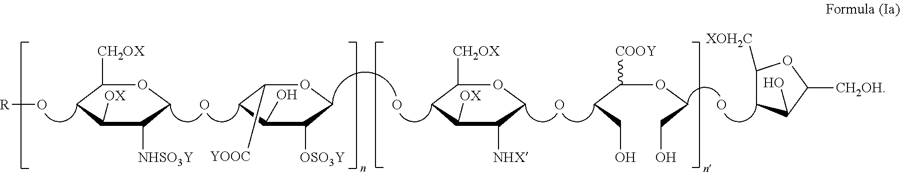

In some embodiments, Y for each occurrence is Na⁺.

In some embodiments, R is

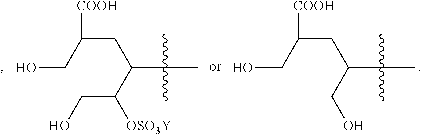

In some embodiments, the polysaccharide of Formula (I) is a polysaccharide of Formula (Ib)

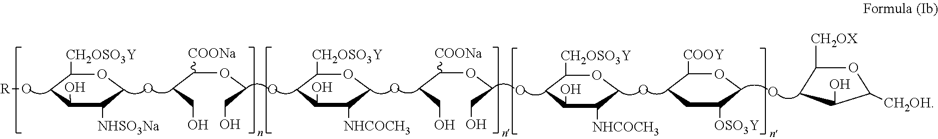

In some embodiments, Y for each occurrence is Na+.

In some embodiments, R is,

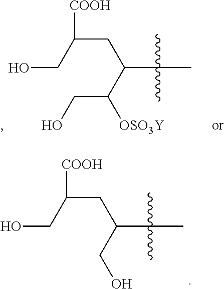

In some embodiments, the preparation consists essentially of polysaccharides having the structure of Formula (I) or Formula (Ia) or Formula (Ib). In some embodiments, the preparation consists of polysaccharides having the structure of Formula (I) or Formula (Ia) or Formula (Ib). In some embodiments, at least about 20% (e.g., at least about 30%, at least about 40%, at least about 50%, at least about 60%, at least about 70%, at least about 80%, at least about 90%, at least about 95%, at least about 98%, or at least about 99%) of the polysaccharides in the preparation have the structure of Formula (I) or Formula (Ia).

Preferably, the preparation has anti-Xa activity of less than 50 IU/mg, 40 IU/mg, 30 IU/mg, 20 IU/mg or 10 IU/mg but greater than 0.5 IU/mg, 1 IU/mg and/or anti-IIa activity of less than 50 IU/mg, 40 IU/mg, 30 IU/mg, 20 IU/mg or 10 IU/mg but greater than 0.5 IU/mg, 1 IU/mg. In some embodiments, the preparation has a weight average chain molecular weight between 3,500 and 8,000 Da, e.g., between 4,000 and 8000 Da, 4,500 and 8,000 Da, 4,700 and 8,000 Da and 5,000 and 8,000 Da. In some embodiments, the preparation has a molecular weight distribution such that 10-50% (e.g., 10-40%, 10-30%, 15-30% or 15-25%) of the oligosaccharides of the preparation have a molecular weight <3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight >8000 Da. The LMWH preparations described herein (e.g., described above) can also be a pharmaceutically acceptable salt of any of the LMWH preparations described herein.

Any of the preparations described herein, e.g., described above, can have other properties. E.g., one of the above described preparations can further have one or more of the functional or structural properties set out below:

the preparation or pharmaceutical preparation has a molecular weight distribution such that 10-50% (e.g., 10-40%, 10-30%, 15-30% or 15-25%) of the oligosaccharides of the preparation have a molecular weight <3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight >8000 Da;

the preparation has a polydispersity of about 1.2 to 1.7 (e.g., about 1.3 to 1.7, 1.4 to 1.6, or 1.3 to 1.6);

the preparation has a polydispersity of about 1.2 to 1.8 (e.g., about 1.3 to 1.8, 1.4 to 1.7, or 1.3 to 1.7);

the preparation has a polydispersity of about 1.2 to 1.9 (e.g., about 1.3 to 1.9, 1.4 to 1.9, or 1.5 to 1.7);

the preparation or preparation has a sodium content less than 30%, 25%, 20%, 15%, 10%. In one embodiment, the preparation or preparation comprises: less than 20 ppm, 15 ppm, 10 ppm, 5 ppm iodine; less than 30%, 25%, 20%, 15%, 10% sulfur; less than 50, 40, 30, 20, 15 ppm boron;

the preparation or preparation has anti-metastatic activity;

the preparation or preparation binds specifically to or inhibits an activity of one or more of: VEGF, FGF, SDF-1-α, HB-EGF, heparanase, SCF, sonic hedgehog, osteopontin, osteopontegerin or P-selectin.

In some embodiments, the LMWH preparation is an M402 preparation. An “M402 preparation” refers to a LMWH preparation that consists essentially of:

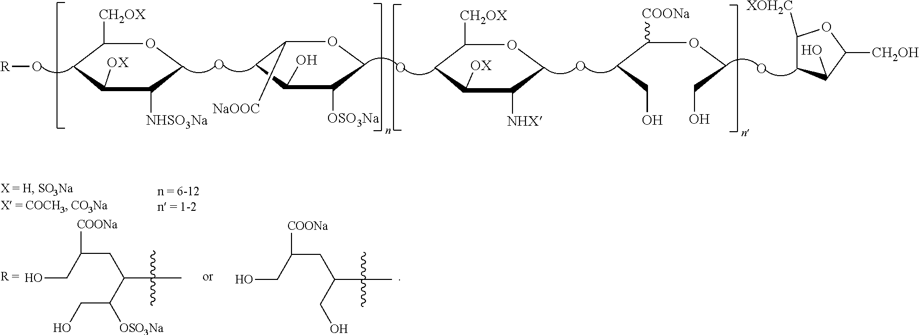

which is also represented as:

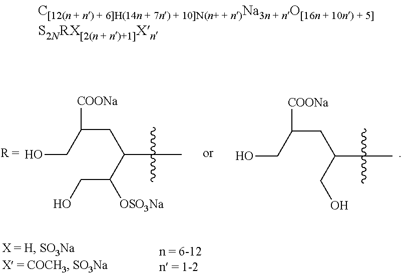

In some embodiments, the M402 preparation is Necuparinol.

In some embodiments, compositions described herein, e.g., LMWH, e.g., glycol split LMWH, are formulated with one or more buffering agent, including but not limited to: a citrate buffer, a phosphate buffer, a histidine buffer, a maleate buffer, a succinate buffer, an acetate buffer, a malate buffer, or any combination thereof.

The pharmaceutical compositions described herein can have a shelf life of at least 30 days, e.g., at least two months, at least three months, at least six months, at least nine months, twelve months, or at least eighteen months).

Any preparation or preparation described herein can be manufactured using good manufacturing practices (GMP) as defined by the U.S. Food and Drug Administration (21 CFR Part 110).

### Anti-IIa Activity

LMWH preparations are disclosed herein that provide substantially reduced anti-IIa activity, e.g., e.g., anti-IIa activity of about less than about 50 IU/mg, less than about 40 IU/mg, 30 IU/mg, 20 IU/mg, 15 IU/mg, 10 IU/mg, 5 IU/mg, 4 IU/mg, 3 IU/mg, 2 IU/mg or 1 IU/mg; or from about 0 to 50 IU/mg, about 0 to 40 IU/mg, about 0 to 30 IU/mg, about 0 to 25 IU/mg, about 0 to 20 IU/mg, about 0 to 10 IU/mg, about 0 to 5 IU/mg, about 5 to 10 IU/mg, about 5 to 15 IU/mg, or about 5 to 20 IU/mg). Anti-IIa activity is calculated in International Units of anti-IIa activity per milligram using statistical methods for parallel line assays. The anti-IIa activity levels described herein are measured using the following principle.

Polysaccharide (PS)+ATIII→[PS·ATIII] IIa

PS·ATIII→[PS·ATIII·IIa]+IIa (Excess)

Ha (Excess)+Substrate→Peptide+pNA (measured spectrophotometrically)

Anti-factor IIa activity is determined by the sample potentiating effect on antithrombin (ATIII) in the inhibition of thrombin. Thrombin excess can be indirectly spectrophotometrically measured. The anti-factor IIa activity can be measured, e.g., on a Diagnostica Stago analyzer or on an ACL Futura3 Coagulation system, with reagents from Chromogenix (S-2238 substrate, Thrombin (53 nkat/vial), and Antithrombin), or on any equivalent system. Analyzer response is calibrated using the 2nd International Standard for Low Molecular Weight Heparin.

### Anti-Xa Activity

Preferably, a LMWH preparation provided herein has anti-Xa activity of about 0 to 50 IU/mg, e.g., 50 IU/mg, 40 IU/mg, 30 IU/mg, 20 IU/mg, 15 IU/mg, 10 IU/mg, 5 IU/mg, 4 IU/mg, 3 IU/mg, 2 IU/mg or 1 IU/mg; or from about 0 to 50 IU/mg, about 0 to 40 IU/mg, about 0 to 30 IU/mg, about 0 to 25 IU/mg, about 0 to 20 IU/mg, about 0 to 10 IU/mg, about 0 to 5 IU/mg, about 5 to 10 IU/mg, about 5 to 15 IU/mg, or about 5 to 20 IU/mg). Anti-Xa activity of a preparation is calculated in International Units of anti-factor Xa activity per milligram using statistical methods for parallel line assays. The anti-factor Xa activity of preparations described herein is measured using the following principle:

PS+ATIII→[PS·ATIII] FXa

PS·ATIII→[PS·ATIII·FXa]+FXa(Excess)

FXa (Excess)+Substrate→Peptide+pNA (measured spectrophotometrically)

The anti-factor Xa activity is determined by the sample potentiating effect on antithrombin (ATIII) in the inhibition of activated Factor Xa (FXa). Factor Xa excess can be indirectly spectrophotometrically measured. Anti-factor Xa activity can be measured, e.g., on a Diagnostica Stago analyzer with the Stachrom® Heparin Test kit, on an ACL Futura3 Coagulation system with the Coatest® Heparin Kit from Chromogenix, or on any equivalent system. Analyzer response can be calibrated using the NIBSC International Standard for Low Molecular Weight Heparin.

### Molecular Weight and Chain Length

LMWH preparations can have a weight average molecular weight described herein.

“Weight average molecular weight” as used herein refers to the weight average in daltons of chains of uronic acid/hexosamine disaccharide repeats. The presence of non-uronic acid and/or non-hexosamine building blocks are not included in determining the weight average molecular weight. Thus, the molecular weight of non-uronic acid and non-hexosamine building blocks within a chain or chains in the preparation should not be included in determining the weight average molecular weight. The weight average molecular weight (M_{w}) is calculated from the following equation: M_{w}=Σ(cᵢmᵢ)/Σcᵢ. The variable cᵢ is the concentration of the polymer in slice i and mᵢ is the molecular weight of the polymer in slice i. The summations are taken over a chromatographic peak, which contains many slices of data. A slice of data can be pictured as a vertical line on a plot of chromatographic peak versus time. The elution peak can therefore be divided into many slices. The weight average molecular weight calculation is average dependant on the summation of all slices of the concentration and molecular weight. The weight average molar weight can be measured, e.g., using the Wyatt Astra software or any appropriate software. The weight average molecular weights described herein are determined by high liquid chromatography with two columns in series, for example a TSK G3000 SWXL and a G2000 SWXL, coupled with a UV or multi angle light scattering (MALS) detector and a refractometric detector in series. The eluent used is a 0.2 M sodium sulfate, pH 5.0, and a flow rate of 0.5 mL/min.

A determination of whether a LMWH preparation includes chains of sufficient chain length can be made, for example, by determining the average chain length of the chains in the preparation and/or by determining the weight average molecular weight of chains within the preparation. For example, when weight average molecular weight of a preparation is determined, a weight average molecular weight of about 3500 to 8000 Da, preferably about 4000 to 8000 Da, about 4200 to 8000, or about 4500 to 8000 Da, indicates that a significant number of chains in the polysaccharide preparation are of a chain length described herein, e.g., for M402, n+n′ has an average chain length of 7-14.

“Average chain length” as used herein refers to the average chain length of uronic acid/hexosamine disaccharide repeats that occur within a chain. The presence of non-uronic acid and/or non-hexosamine building blocks (e.g., attached PEG moieties) are not included in determining the average chain length. Average chain length is determined by dividing the number average molecular weight (Mn) by the number average molecular weight for a disaccharide (500 Da).

### Molecular Weight Distribution

The molecular weight distribution of a LMWH preparation described herein can be determined by known methods.

In some embodiments, a LMWH preparation described herein has a molecular weight distribution such that 10-50% (e.g., 10-40%, 10-30%, 15-30% or 15-25%) of the oligosaccharides of the preparation have a molecular weight <3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight >8000 Da. In certain embodiments, a LMWH preparation described herein has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

### Glycol Split Uronic Acids

A LMWH preparation described herein can include an opening of the glycoside ring, conventionally called reduction-oxidation (RO) derivatives. In these preparations, one or more glycoside rings having vicinyl diols that are opened, e.g., at the bond between C2 and C3, by means of an oxidation action, followed by a reduction. The compounds referred to herein will also be called “Glycol Split” derivatives.

In some embodiments, the glycol split residues lend themselves to the subsequent functionalization. Therefore, the compounds may also bear equal or different groups, in place of the primary hydroxy groups deriving from glycol split, for example, aldehyde groups, methoxy groups, or oligosaccharide or peptide groups, ranging from a single saccharide or amino acid to more than one unit of length, e.g., 2 or 3 units.

In some embodiments, fewer than 50% of the uronic acid residues are glycol split uronic acid residues (e.g., less than 40%, 30%, 25%, or 20% of the uronic acid residues are glycol split uronic acid residues).

### Reducing End Structures

In some instances, at least about 50% of the chains in a LMWH preparation described herein have a modified reducing end structure such as a 2,5-anhydromannose residue or a 2,5-anhydromannose that has been reduced to form an alcohol. In some embodiments, at least about 55%, 60%, 65%, 70%, 75%, 80%, 85%, 90%, or 95% of the chains in the preparation have a modified reducing end structure, such that the reducing end includes a 2,5-anhydromannose residue or a 2,5-anhydromannitol.

### Polydispersity

The polydispersity of LMWH preparations provided herein is about 2 or less, e.g., 1.7 or less, e.g., about 1.9, 1.8, 1.7 or 1.6 to 1.2, about 1.4-1.5, and numbers in between.

The term “polydisperse” or “polydispersity” refers to the weight average molecular weight of a preparation (Mw) divided by the number average molecular weight (Mn). The number average molecular weight (Mn) is calculated from the following equation: Mn=Σci/(Σci/mi). The variable ci is the concentration of the polysaccharide in slice i and Mi is the molecular weight of the polysaccharide in slice i. The summations are taken over a chromatographic peak, which contains many slices of data. A slice of data can be pictured as a vertical line on a plot of chromatographic peak versus time. The elution peak can therefore be divided into many slices. The number average molecular weight is a calculation dependent on the molecular weight and concentration at each slice of data. Methods of determining weight average molecular weight are described above, and were used to determine polydispersity as well.

### Methods of Making LMWH Preparations

The LMWH preparation can be made, e.g., by known methods. In some embodiments, a LMWH preparation lacking substantial anticoagulant activity can be made by a method that includes providing a precursor polysaccharide preparation having a weight average molecular weight of greater than 7000 Da or a chain length of greater than 7 to 18 disaccharides, and processing the precursor polysaccharide preparation (e.g., by enzymatic or chemical depolymerization, e.g., by nitrous acid depolymerization) to obtain a polysaccharide preparation having a weight average molecular weight of about 3000 to 8000 Da or an average chain length of about 7 to 18 disaccharides. For example, the precursor polysaccharide preparation can be unfractionated heparin.

The precursor polysaccharide preparation can be processed by a method comprising depolymerization (e.g., by nitrous acid treatment, hydrolysis, or enzymatic depolymerization) followed by a glycol split reaction. Nitrous acid depolymerization can be accomplished, e.g., by treating the precursor polysaccharide preparation (e.g., UFH) with nitrous acid (e.g., about 0.02 to 0.04 M nitrous acid) at a pH of about 2 to 4 for a specified period of time (e.g., about 1 to 5 hours) at a temperature of about 10 to 30° C. The glycol split reaction involves periodate oxidation using periodate (e.g., about 0.05 M to 0.2 M sodium periodate) for about 10 to 20 hours at a temperature of about 0 to 10° C. In some embodiments, residual impurities such as salts or diethylene glycol (DEG) can be subsequently removed by a chromatographic method, e.g. gel filtration chromatography. Optionally, the oxidized preparation is then reduced by treatment with a reducing agent (e.g., about 0.5 to 2.0% (w/v) sodium borohydride) for about 0.5 to 3 hours at a pH of about 6.0 to 7.0 and a temperature of about 0 to 10° C.

A precursor polysaccharide preparation can be processed using enzymatic digestion, chemical digestion or combinations thereof. Examples of chemical digestion include oxidative depolymerization, e.g., with H₂O₂ or Cu⁺ and H₂O₂, deaminative cleavage, e.g., with isoamyl nitrite or nitrous acid, β-eliminative cleavage, e.g., with benzyl ester, and/or by alkaline treatment. Enzymatic digestion can include the use of one or more heparin degrading enzymes. For example, the heparin degrading enzyme(s) can be, e.g., one or more heparanase, heparin lyase, heparan sulfate glycoaminoglycan (HSGAG) lyase, a lyase described as a glycoaminoglycan (GAG) lyase that can also degrade heparin.

### Structural Signatures

Exemplary structural signatures include one or both of formic acid and C₂H₆O₃. In some embodiments of the aspects described herein, the structural signature is indicative of the stability of the LMWH preparation, e.g., of the LMWH preparation in a pharmaceutical composition.

Exemplary structural signatures include, but are not limited to, formic acid and/or

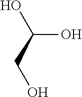

(C₂H₆O₃). In one embodiment, when a LMWH preparation is substantially free of formic acid and/or

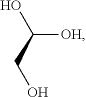

the LMWH preparation is a stable preparation. In some embodiments, the LMWH preparation contains less than (e.g., 10%, 9%, 8%, 7%, 6%, 5%, 4%, 3%, 2%, 1%, 0.5%, 0.3%, 0.2%, 0.1%, 0.05%) of formic acid as determined by 1D NMR and/or

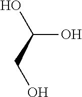

as determined by 2D NMR. In some embodiments, the LMWH preparation contains between 0.0001-2% (e.g., 0.001-2%, 0.01-2%, 0.1-2%, 1-2%, 0.05-2%, 0.5-2%, 0.5-1%) of formic acid as determined by 1D NMR and/or

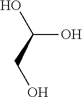

as determined by 2D NMR. In some embodiments, the LMWH preparation contains less than (e.g., 10%, 9%, 8%, 7%, 6%, 5%, 4%, 3%, 2%, 1%, 0.5%, 0.3%, 0.2%, 0.1%, 0.05%) of formic acid as determined by 1D NMR and/or

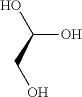

as determined by 2D NMR, when stored at 4° C. (e.g., 2-6° C., 3-4° C., 3-6° C., 4-5° C., 4-6° C.) for 3 months or more, (e.g., 4, 5, 6, 7, 8, 9, 10, 11, 12, 24, 36 months or more). In some embodiments, the LMWH preparation contains between 0.0001-2% (e.g., 0.001-2%, 0.01-2%, 0.1-2%, 1-2%, 0.05-2%, 0.5-2%, 0.5-1%) of formic acid as determined by 1D NMR and/or

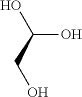

as determined by 2D NMR, when stored at 4° C. (e.g., 2-6° C., 3-4° C., 3-6° C., 4-5° C., 4-6° C.) for 3 months or more, (e.g., 4, 5, 6, 7, 8, 9, 10, 11, 12, 24, 36 months or more).

### Analytical Techniques

Analytical techniques described herein can include, but are not limited to, one or more of, or any combination of the following: a spectroscopy method, e.g., a nuclear magnetic resonance (NMR) method, e.g., 1D NMR, e.g., ¹H NMR, 2D NMR, e.g., 2D homonuclear NMR, 2D heteronuclear NMR, e.g., heteronuclear single quantum coherence spectroscopy (HSQC.

### Packaging/Kits/Research Tools

Also disclosed herein are LMWH preparations (e.g., a LMWH preparation described herein) which are packaged for administration to a human subject. The packaging can be associated with a label. The label can indicate, inter alia, a value of the level of formic acid and/or a value of the level of C₂H₆O₃. The packaging can be associated with instrunctions. The instrunctions can indicate, inter alia, methods of preparing the LMWH preparation for administration to a human subject and/or methods of administering the LMWH preparation to a human subject. Also disclosed herein are methods of advising on, or providing instructions (e.g., written, oral, or computer generated instructions (e.g., instructions described herein)) for the use of a LMWH preparation described herein.

Also disclosed herein are kits comprising a LMWH preparation described herein. The kit can include one or more other elements including: instructions (e.g., written, oral, or computer generated instructions (e.g., instructions described herein)) for the use of a LMWH preparation described herein. The kit can also include other reagents, devices, or other materials for preparing the LMWH preparation for administration to a human subject; pharmaceutically acceptable carriers; and devices or other materials for administration of the LMWH preparation to a human subject.

### EXEMPLIFICATION

### EXAMPLE 1

### M402 Analysis via NMR

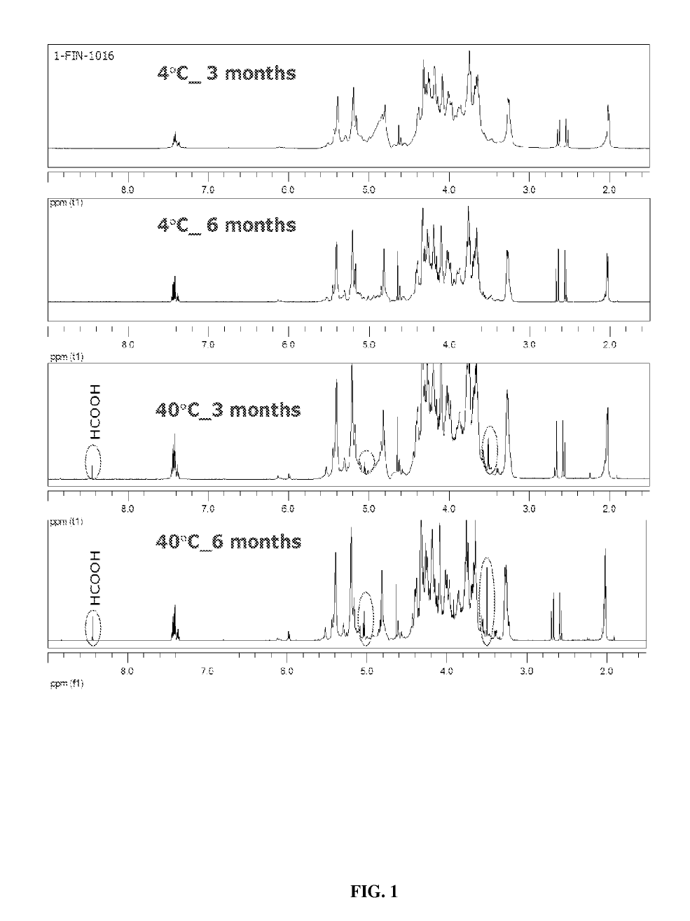

The LMWH designated herein as M402 was stored at different storage conditions selected to accelerate any degradation of M402, including 4-5° C., 25° C./60% Relative Humidity (RH), 30° C./65% RH, and 40° C./75% RH for three or six months. After three or six months of storage, samples were analyzed by ¹H-NMR and 2D-NMR. As shown in FIG. 1 samples stored at 40° C./75% RH present additional signals, which were assigned to formic acid (HCOOC) and to a small structure released during degradation of glycol-split signals:

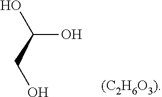

Overall, NMR analysis detects major structural changes in M402 samples stored at 40° C./75% RH, in particular a release of formic acid and LMW residues.

1. A pharmaceutical composition comprising a LMWH preparation wherein the LMWH preparation has the following characteristics:
(a) a weight average chain molecular weight between 3,500 and 8,000 Da;
(b) anti-Xa activity of less than 20 IU/mg and anti-IIa activity of 20 IU/mg or less;
(c) greater than 5% and less than 50% glycol split uronic acid residue (U_{G}) in the preparation, and wherein formic acid is detectable in the composition but at less than 2%, 1%, 0.5%, 0.3%, 0.2%, 0.1%, or 0.05% as determined by 1D-NMRand/or C₂H₆O₃ is detectable in the composition but at less than 2%, 1%, 0.5%, 0.3%, 0.2%, 0.1%, or 0.05% as determined by 2D-NMR.

2. The pharmaceutical composition of claim 1, wherein formic acid is detectable in the composition at a level between 0.0001-2% and/or C₂H₆O₃ is detectable in the composition at a level between 0.0001-2%.

3. The pharmaceutical composition of claim 1, wherein the LMWH preparation has an
anti-IIa activity of 1 U/mg or less;

4. The pharmaceutical composition of claim 1, wherein the LMWH preparation contains less than 1%, less than 0.5%, or less than 0.1% of formic acid when stored at about 4° C. for 3 months or more.

5. The pharmaceutical composition of claim 1, wherein the LMWH preparation is an M402 preparation.

6. The pharmaceutical composition of claim 1, wherein the M402 preparation is necuparanib.

7. The pharmaceutical composition of claim 1, wherein the LMWH preparation comprises a polysaccharide of Formula (I) 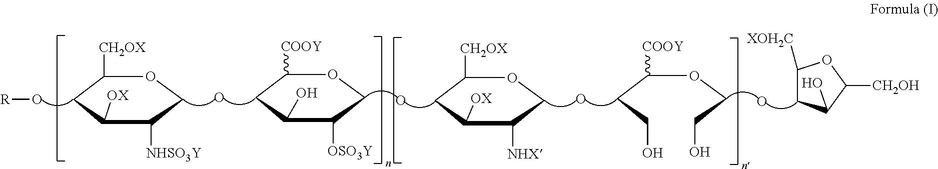
wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and
R is

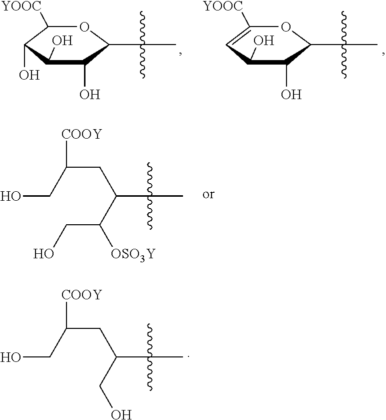

8. The pharmaceutical composition of claim 7, wherein the compound of Formula (I) is a compound of Formula (Ia)

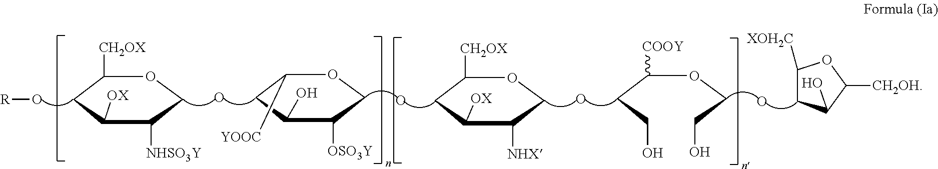

9. The pharmaceutical composition of claim 7, wherein Y for each occurrence is Na⁺.

10. The pharmaceutical composition of claim 7, wherein, R is

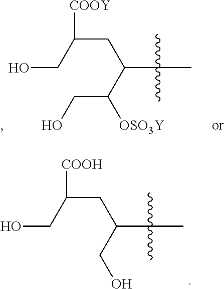

11. The pharmaceutical composition of claim 1, wherein the LMWH preparation comprises or consists essentially of:

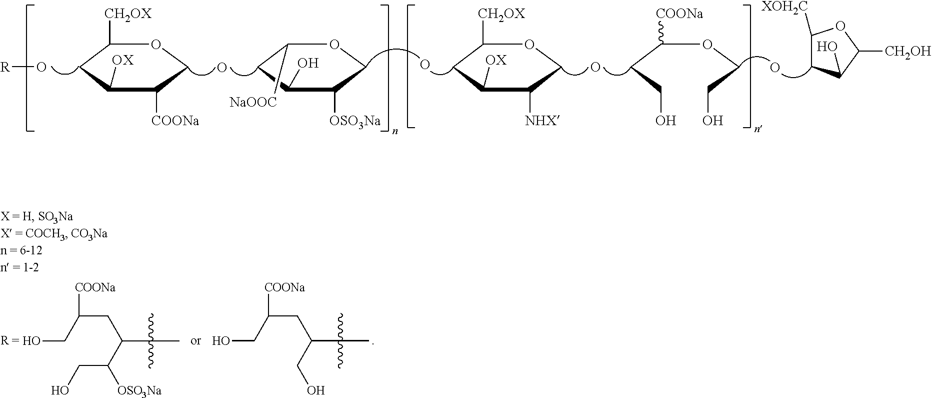

12.-14. (canceled)

15. The pharmaceutical composition of claim 1, wherein the level of C₂H₆O₃ is determined by 2D-NMR.

16. The pharmaceutical composition of claim 1, wherein the level of formic acid is determined by 1D-NMR technique.

17.-23. (canceled)

24. A method of producing a pharmaceutical composition comprising a LMWH preparation having at least one chain having a glycol split uronic acid residue (U_{G}), the method comprising:
providing a LMWH preparation having at least one chain having a glycol split uronic acid residue (U_{G}) in the preparation;
determining, by performing an analytical technique the presence or absence of a structural signature; and
if the structural signature is present in an amount that indicates that the preparation is stable, formulating the LMWH preparation, to thereby produce the pharmaceutical composition.

25. The method of claim 24, wherein the LMWH preparation has the following characteristics:
(a) a weight average chain molecular weight between 3,500 and 8,000 Da;
(b) anti-Xa activity of less than 20 IU/mg and anti-IIa activity of less than 20 IU/mg;
(c) greater than 5% and less than 25% glycol split uronic acid residues; and
(d) the polysaccharide preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.

26.-36. (canceled)

37. A method of manufacturing an M402 preparation, the method comprising:
(a) obtaining a preparation of unfractionated heparin (UFH);
(b) depolymerizing the UFH for a time and under conditions to obtain a first polysaccharide preparation having a weight average molecular weight of 3000-8000 Da;
(c) glycol splitting the first polysaccharide preparation to obtain a second polysaccharide preparation;
(d) reducing and purifying the second polysaccharide preparation;
(e) measuring formic acid and/or C₂H₆O₃ in the reduced and purified second polysaccharide preparation; and
(f) processing the second polysaccharide preparation as M402 drug product or drug substance if formic acid is less than 2%, 1%, 0.5%, 0.3%, 0.2%, 0.1%, 0.05% as determined by 1D NMR and/or C₂H₆O₃ is less than 2%, 1%, 0.5%, 0.3%, 0.2%, 0.1%, 0.05% as determined by 2D NMR,
to thereby manufacture an M402 preparation.

38.-43. (canceled)

44. The pharmaceutical composition of claim 1, wherein the LMWH preparation has a molecular weight distribution such that 10-40% of the oligosaccharides of the preparation have a molecular weight <3000 Da; 45-65% of the oligosaccharides have a molecular weight between 3000-8000 Da, and 15-30% of the oligosaccharides have a molecular weight >8000 Da.
